(19) United States
(12) Patent Application Publication
(10) Pub. No.: US 2014/0283826 A1
(43) Pub. Date: Sep. 25, 2014

(54) Quick Release Magnetic Clasp for Continuous Positive Airway Pressure Interface Devices

(71) Applicants: Curtis Murray, San Antonio, TX (US); Lisa M. Jackson, San Antonio, TX (US)

(72) Inventors: Curtis Murray, San Antonio, TX (US); Lisa M. Jackson, San Antonio, TX (US)

(21) Appl. No.: 14/219,441

(22) Filed: Mar. 19, 2014

Related U.S. Application Data
(60) Provisional application No. 61/803,323, filed on Mar. 19, 2013.
(60) Provisional application No. 61/803,604, filed on Mar. 20, 2013.

(51) Int. Cl. A61M 16/06

(52) U.S. Cl. CPC A61M 16/0683
USPC 128/202.27

(57) ABSTRACT

A respiratory mask for Continuous Positive Airway Pressure (CPAP) therapy. The device is suitable for use by the elderly and/or persons of limited dexterity who otherwise may not be able to effectively manipulate standard attachment devices of the type found on commonly available CPAP masks. The device utilizes both magnetic attraction and a receiver slot and hook method of binding one connector to a mating receiving connector. The joint between the two portions acts as a rotating pivot assembly that can disengage the device working as a simple lever once the release spur is actuated.

### CLAIM OF PRIORITY TO PRIOR APPLICATIONS

This application claims the benefit of U.S. Provisional Application Ser. No. 61/803,323, filed Mar. 19, 2013, and U.S. Provisional Application Ser. No. 61/803,604, filed Mar. 20, 2013, both entitled “Quick Release Magnetic Clasp for Continuous Positive Airway Pressure Interface Devices”, the entire disclosures of which are hereby incorporated by reference into the present disclosure.

### STATEMENT REGARDING FEDERALLY FUNDED RESEARCH AND DEVELOPMENT

The inventions described and claimed in this patent application were not the subject of federally sponsored research or development.

### FIELD OF THE INVENTION

This invention relates to Continuous Positive Airway Pressure (CPAP) masks. More particularly, this invention relates to CPAP masks which can quickly be released or opened to remove the mask and just as quickly replaced by individuals with limited dexterity due to medical conditions.

### BACKGROUND

CPAP masks used for the therapeutic delivery of oxygen or other gases to a patient can be irritating and uncomfortable to the patient as well as induce feelings of claustrophobia. Additionally, patients who are required to wear a mask for an extended time period, as in CPAP therapy, may need to remove the mask quickly as when experiencing aerophagia, vomiting or panic attacks. CPAP therapy is the most recommended and the most effective treatment for Obstructive Sleep Apnea. A CPAP machine provides air at a constant prescribed pressure through a tube to the CPAP mask. The mask is held in place with a harness of varying styles. The harness and mask attach to each other with the use of hook latch fabric and pressure release clasps. In many instances, out of necessity, if not expediency, it is required that the clasp be activated or released quickly. In many scenarios, the patient is outside of a hospital setting, often at home and may have diminished dexterity issues due to age or medical conditions.

Currently available delivery masks of the type used for CPAP therapy consist of a mask which is held in place against the patient's face with a strap harness of varying styles. The straps are attached to the mask most commonly via a clasping mechanism located at or near the interface of the mask with the strap. Commonly available clasping mechanisms may be of a buckle type which requires depression of a set of opposed locking lugs or may include a spring loaded button which fits into a receptacle on the mating strap or at the mask. Such mechanisms contribute to patient noncompliance as they may difficult for a person of limited dexterity to operate in an urgent situation and for this reason, the patient may be afraid to sleep with the CPAP mask on.

A need has long existed for a CPAP mask readily usable by persons with limited dexterity due to disability, disease, injury, youth, or advanced age. Such persons often find it difficult or painful to manipulate CPCP buckles or clasps and, as a result, resist use of the CPCP masks.

### SUMMARY OF THE INVENTION

The present invention provides for a CPAP mask with a closure system requiring minimal skill and dexterity for interconnecting the mask to the headgear required to keep it in place on the patient. The closure system provides an easy to operate mechanism which is quick to release and reattach by those at greatest risk of non-compliance in CPAP or other therapies.

Such a mask and closure system is inherently more efficient and easy to use than prior teaching in the art. Because of this, the invention is safer and more effective than prior art, resulting in vastly increased patient compliance and an overall better CPAP mask. In its most basic form, a clasp means is formed comprising a base unit acting as a catch connected to the headgear and a hook arm connected to the mask assembly. The embodiments of the invention utilize a magnetic closure mechanism to connect the CPAP mask to the headgear in order to keep it in place. Some embodiments of the invention utilize an interconnecting magnetic adaptor that may be fitted to other clasps on common commercially available CPAP masks in order to convert their mechanical clasps into a magnetic quick release closure.

### BRIEF DESCRIPTION OF THE DRAWINGS

A better understanding of the present inventions and some of the corresponding embodiments may be had by reference to the drawing figures wherein:

FIG. 1 is a perspective top view of a preferred embodiment of the CPAP clasp while in the closed configuration;

FIG. 2 is a perspective top view of the device shown in FIG. 1 in an open configuration;

FIG. 3 is a perspective side view of the device shown in FIG. 1 in an open configuration;

FIG. 4 is a perspective side view of the device male mating arm shown in FIG. 1 and 2;

FIG. 5 is a perspective side view of the device female receiver base unit shown in FIG. 3 showing internal component layout;

FIG. 6 is a functional view illustrating the preferred embodiment in use in the closed position;

FIG. 7 is a functional view illustrating the preferred embodiment in use in the open position; and

FIG. 8 is a functional view illustrating an example of prior art.

### DETAILED DESCRIPTION OF PREFERRED EMBODIMENT

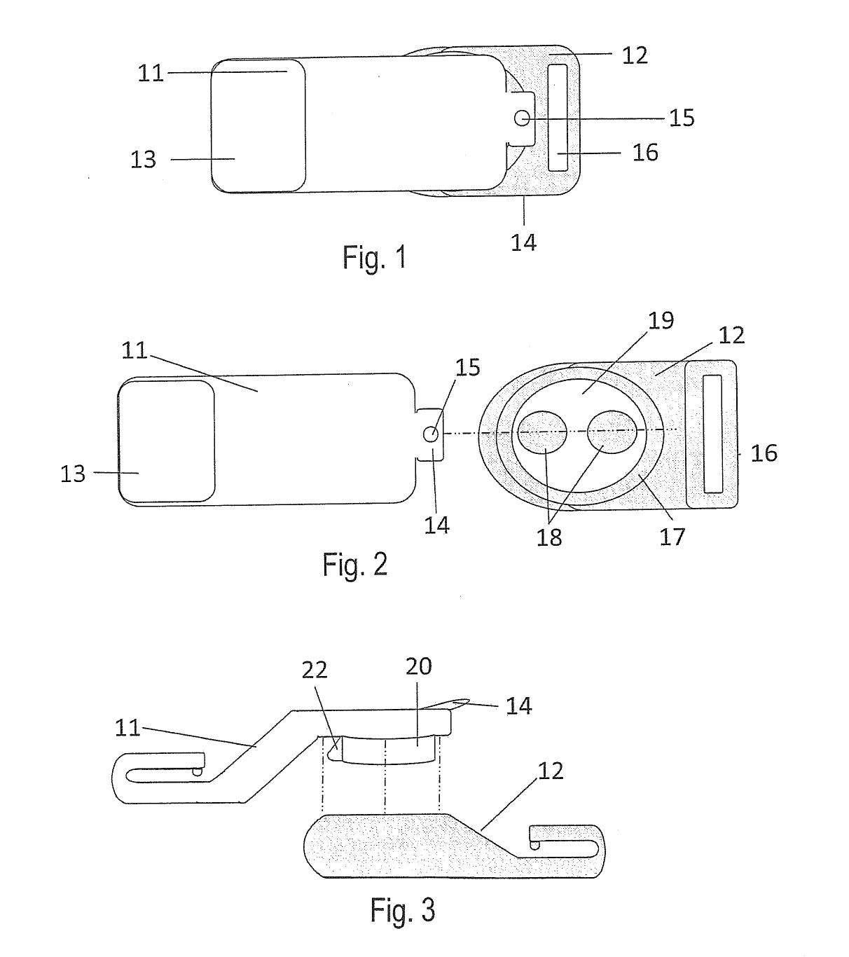

As shown in the accompanying drawings and referring here to FIGS. 1-3, the buckle assembly is comprised of a set of connectors 11 and 12, where 11 is the device mating arm and 12 is the device receiver base. Each connector is comprised of a magnetic mating surface end comprising a mating female receptacle seat 19 and a protruding male device arm magnet 20 with attachment point ends 13 and 16 to receive the strap or connector of a CPAP or other mask. The connector units are molded from a resilient plastic material such as, for example, nylon or polypropylene.

Referring now to FIG. 3, the mating surface end of connector 11 is comprised of a protruding magnet 20 of medium tensile strength, which is permanently attached to the mating surface. The protruding magnet is aligned so that it is perpendicular to the longitudinal axis of the mating arm 11. In the illustrated embodiment the protruding magnet 20 and associated receptacle 19 are of a circular disk configuration, but it would be understood by persons of skill in the art that other configurations may be adapted for this function in a similar fashion.

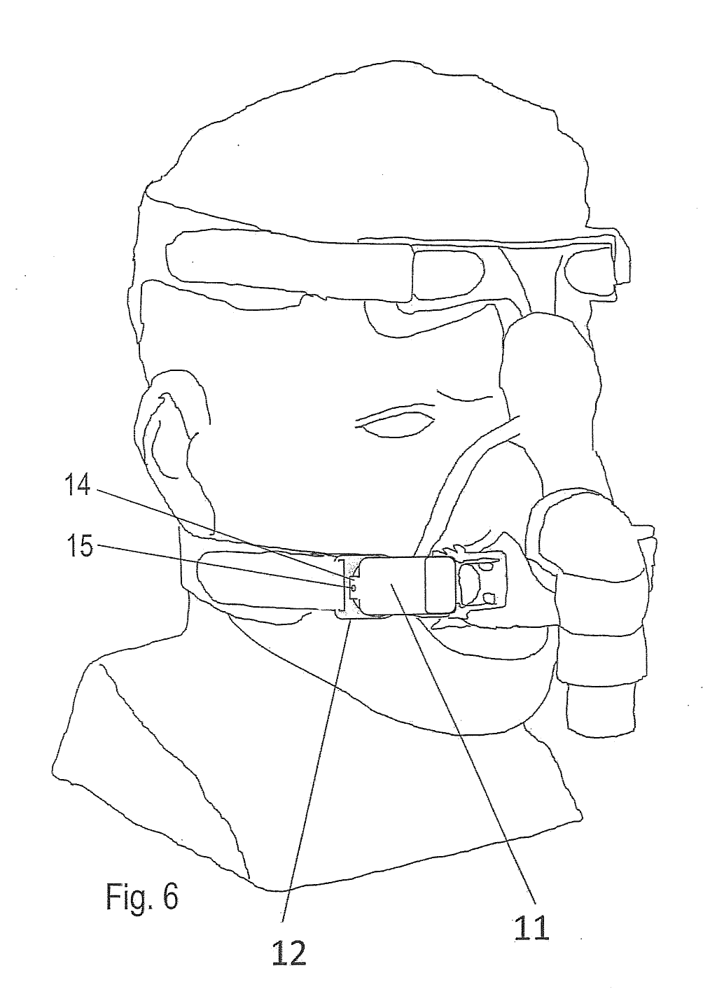

Referring to FIG. 5, the protruding magnet 20 is designed so as to insert into the receptacle of connector 12 via a mating receptacle seat 19 which is molded into the receiving connector base at one end of the connector 12 and is shaped as the converse of protrusion 20. The connector base receptacle is designed so that the longitudinal axis of the disk shaped void is likewise perpendicular to the longitudinal axis of the connector 12. A magnet or magnets 18 are contained within the receiving connector base 12 and positioned at the base of mating receptacle seat 19 so as to attract the magnet 20 of mating arm connector 11 when the mating surfaces of 19 and 20 are moved in proximity to each other. The attractive force of magnets 18 and 20 is sufficient to retain the combined connector assembly as an integral unit while the CPAP or other ventilation device is in operation. In the illustrated embodiment there is shown one magnet for 20 and 2 magnets for 18, but it would be immediately apparent to any person of skill in the art that other numbers and configurations of magnets would perform similarly.

Referring again to FIGS. 1-3, molded within the ends of the mating arm connector 11 and receiver connector base 12 components are slots, channels or other means (13, 16) for the attachment of the strap ends of a CPAP or other mask strap. In the illustrated embodiment, strap ends are inserted into slots 13 and 16 of the component parts 11 and 12 of the invention and secured by hook and loop or other fastening means common to such mask straps. In the preferred embodiment, the invention functions as a universal replacement for the original mask connecting clasp mechanism by removing the original mask connecting clasp mechanism and inserting the freed strap ends into slots 13 and 16 of the invention. In other embodiments, the invention is supplied as an integral part of a commercially available CPAP mask or has connectors which otherwise attach to and secure the invention to the original mechanical connection mechanism of a commercially available mask.

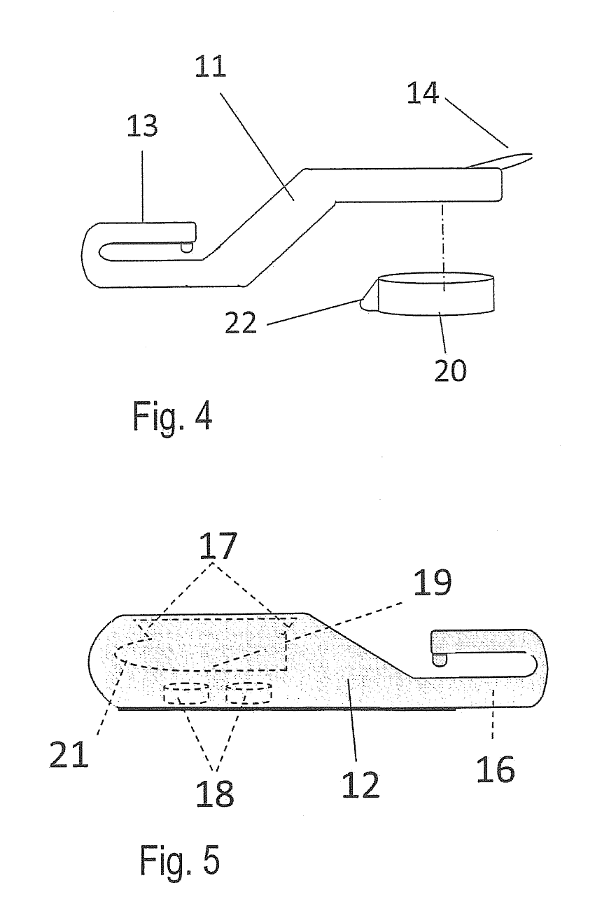

Referring to FIGS. 4 and 5, there exists on mating arm connector 11 a lever release arm 14 by which the patient may obtain additional leverage in separating the protruding mating arm connector 11 and the receiving connector base 12 by inserting a fingernail or applying finger friction so as to effect release of the magnetic clasping action.

To provide an additional means of connection guidance and support, the protruding mating arm connector contains a beveled edge protrusion 22 located adjacent to and continuous with the protruding magnet 20. This beveled edge protrusion slides into a converse receptacle slot 21 which is molded as a recess into the matching opposing side of receptacle connector mating recess 19. Beveled edges 17 located at the extreme peripheral margin of receptacle mating recess 19 provide further guidance assistance in locating connector halves 11 and 12 so that protruding magnet 20 smoothly aligns and inserts into receptacle mating recess receptacle seat 19.

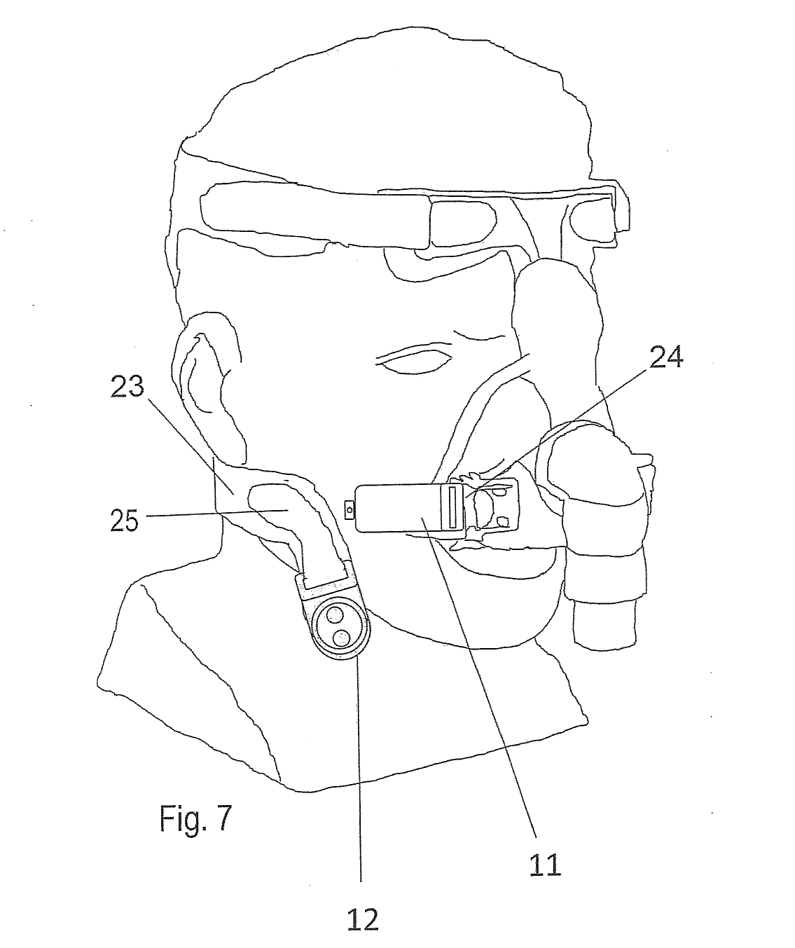

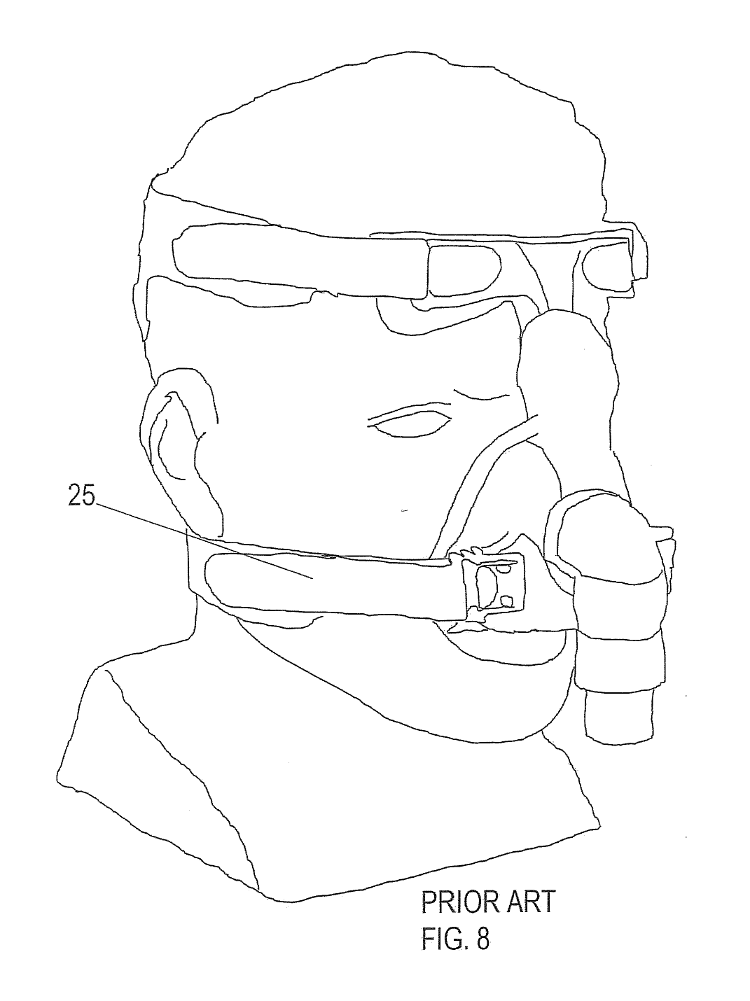

Referring now to FIGS. 6 and 7, the preferred embodiment is shown in an attached configuration. In this embodiment, the invention is attached to the straps of a CPAP mask by first removing the original mechanical connectors supplied with the CPAP mask and inserting the invention components onto either end of the mask straps by way of slots 13 and 16. Such straps are commonly of a hook and loop adjustable variety.

When a patient desires to remove the mask, they apply finger pressure to the release spur 14 or pull a string or other attachment connected to accessory port 15. Such actions serve to separate the two clasping halves 11 and 12 and the lower strap is thus disengaged from the CPAP mask as shown in FIG. 7 so that the patient may quickly remove the mask as, for example, when vomiting. To reapply the mask to the attached configuration of FIG. 6, the patient places the protruding mating arm connector 11 in proximity to receiving connector 12 in such a fashion so that mating arm magnet 20 and mating receptacle seat 19 are roughly aligned. High tensile strength magnets 18 are located in the base of receptacle seat 19, and may be configured so as to be subjacent to the recess surface. These lower receptacle magnets will attract the magnets contained in connector 20 and guide the protruding assembly into the receptacle seat 19. The Input bevel 17 (FIG. 5) acts as a guide to properly align and guide protruding magnet 20 into receptacle seat 19. Once the mating arm connector 11 and the receiver connector base 12 attach magnetically, the hook bevel catch 21 further secures the clasp until it is again disengaged by the operation of the release spur 14 or the release accessory port 15. Once the release spur 14 has been snagged by the user the motion of the lever created by the movement of the hook arm 11 provides the mechanical energy required to separate the magnets in each unit allowing the clasp to be separated.

FIG. 8 illustrates one of several varieties of currently available release mechanisms in position and securing a CPAP mask to the user's face. This example of prior art uses a squeeze clasp mechanism affixed to the lower retaining strap 23 via hook and loop closure 25. Such a clasp is cumbersome and may be difficult for many patients. FIGS. 6 and 7 show an embodiment of the invention affixed to the CPAP mask strap following removal of the original clasp device and application of the invention components to lower strap by inserting and reattaching the hook and loop attachment portion of the lower strap.

In all respects, it should also be understood that the drawings and detailed descriptions of embodiments herein are provided by way of example only and are to be regarded in an illustrative rather than in a restrictive manner. Such drawings and descriptions of the examples are not intended to limit the systems and methods of the present invention. Rather, the present invention includes all articles, systems and processes within the scope and spirit of the invention as claimed, as the claims may be amended, replaced or otherwise modified during the course of related prosecution. Any current, amended, or added claims should be interpreted to embrace all further modifications, changes, rearrangements, substitutions, alternatives, design choices, and embodiments that may be evident to those of skill in the art, whether now known or later discovered. In any case, all substantially equivalent variations should be considered within the scope of the invention and, absent express indication otherwise, all structural or functional equivalents are anticipated to remain within the spirit and scope of the disclosed system and method.

We claim:

1. A Continuous Positive Airway Pressure (CPAP) mask and strap assembly, comprising:
a male protrusion connector and a mating female receptacle connector wherein said male protrusion connector component has at one end a magnet perpendicular to the long axis of the male connector arm and at the opposing end a flange, slot or other means by which to attach said male connector arm to a strap and,
a female receptacle component which at one end has a molded receptacle void, shaped as the converse of the protruding magnet portion of the male protrusion connector and at the other end a flange, slot or other means by which to attach said female receptacle arm to a strap and which molded receptacle portion has magnets embedded within and positioned so as to lie directly subjacent to the base of said molded receptacle and,
wherein the magnetic elements of said male protrusion connector and said female receptacle arm connector are positioned so that when brought into proximity with each other exert an attractive force sufficient to draw the male protruding magnet into the female receptacle and bring the two components together.

2. The structure of claim 1 wherein the magnet protrusion of said male connector is further comprised of a wedge shaped flange extending rearward from said protruding magnet portion along the midline of the connector assembly and wherein the conversely molded receptacle void of the female receptacle connector is further comprised of a wedge shaped void extending forward along the midline of the connector such that when the male and female connector portions are brought into proximity the wedge flange of the male protrusion component inserts into the converse wedge void of the female receptacle and aligns the combined male and female connector as a unit with a defined combined longitudinal axis.
